(19) United States
(12) Patent Application Publication
(10) Pub. No.: US 2005/0217538 A1
(43) Pub. Date: Oct. 6, 2005

(54) Modified Calcium Phosphate Bone Cement

(75) Inventors: Antje Reinstorf, Dresden (DE); Wolfgang Pompe, Hartha (DE); Anne Bernhardt, Dresden (DE); Michael Gelinsky, Dresden (DE); Berthold Niles, Frankisch-Crumbach (DE)

Correspondence Address: GUDRUN E. HUCKETT DRAUDT, LONSSTR. 53, WUPPERTAL, 42289, DE

(73) Assignee: TECHNISCHE UNIVERSITAT DRESDEN, Dresden (DE)

(21) Appl. No.: 10/711,619

(22) Filed: Sep. 29, 2004

(30) Foreign Application Priority Data
Mar. 31, 2004 (DE) 20 2004 005 420.5

(51) Int. Cl. A61F 2/28

(52) U.S. Cl. 106/690; 623/23.62; 424/602

(57) ABSTRACT

A calcium phosphate bone cement setting to a calcium-deficient hydroxyl apatite is modified by an organic phosphate ester of orthophosphoric acid or a salt of an organic phosphate ester. The base cement contains preferably tricalcium phosphate, dicalcium phosphate (anhydrous), calcium carbonate and precipitated hydroxyl apatite. The organic phosphate ester is added to the base cement in an amount of 0.5 to 5 percent by weight. The bone cement can be modified further by adding mineralized collagen I.

### BACKGROUND OF THE INVENTION

The invention relates to a modified calcium phosphate bone cement for use in the medical field (surgery) that is used as a material for filling bone defects (as a temporary bone substitute) and for embedding small implants.

The development of calcium phosphate bone cement (CPBC) has been worked on since the 1980s. There exist numerous mixtures of different calcium phosphate compounds that can be processed with liquids to a paste and are thus suitable for filling bone defects. The bone cements sets (cures) within the body. In this connection, the starting compounds are converted within a few days to bone-like calcium-deficient hydroxyl apatite (CDHAP). This “synthetic” CDHAP is replaced over time with endogenic bone material. This is realized by the metabolic activity of the cells of the surrounding tissue.

A wide field of application of calcium phosphate bone cement is the mouth and jaw area of the face. In this area, very high pressures are often generated so that a sufficiently high strength of the calcium phosphate bone cement (CPBC) is unconditionally required. Moreover, a high specific surface area is desirable in order to enable fast coverage by a plurality of cell-active substances of the blood serum. A conversion of the starting material of the cement mixture while only minimal changes of the ion concentration (protons/pH, calcium ions, phosphate ions) of the surrounding medium occur is essential for culturing cells.

### SUMMARY OF THE INVENTION

It is an object of the invention to modify a calcium phosphate bone cement mixture in such a way that it leads to a material that has an increased strength, a greater specific surface area, and an improved cell and tissue compatibility.

According to the present invention, this is achieved by providing a modified calcium phosphate bone cement that sets to a calcium-deficient hydroxyl apatite, wherein the calcium phosphate bone cement comprises an organic phosphate ester of orthophosphoric acid, preferably a monophosphate ester, or a salt of such an organic phosphate ester. Preferably, the calcium phosphate bone cement comprises a base cement comprised of tricalcium phosphate, dicalcium phosphate (anhydrous), calcium carbonate, and precipitated hydroxyl apatite (PHAP),

The starting mixture (base cement) of the calcium phosphate bone cement contains preferably 50 to 65 percent by weight, particularly preferred 58 percent by weight, alpha-tricalcium phosphate (alpha-TCP); preferably 20 to 30 percent by weight, particularly preferred 24 percent by weight, anhydrous dicalcium phosphate (DCPA); preferably 5 to 12 percent by weight, particularly preferred 8.5 percent by weight, calcium carbonate (CC); and preferably 5 to 12 percent by weight, particularly preferred 8.5 percent by weight, precipitated hydroxyl apatite (PHAP).

The aforementioned starting mixture are preferred formulations for the calcium phosphate bone cement of the present invention. However, these formulations are not to be understood as a limitation of the scope of the invention because a person skilled in the art is aware that calcium phosphate bone cements that are comprised in the set or cured state primarily of calcium-deficient hydroxyl apatite (CDHA) can also be obtained from other compositions. Such variations of the cement composition are expressly encompassed in the scope of the present invention because the principle of controlled setting of the cement is not limited to a special calcium phosphate bone cement formulation but is characteristic of cements that cure or set to CDHA.

To the starting mixture or base cement of the calcium phosphate bone cement, preferably 1 to 10 percent by weight, especially preferred up to 5 percent by weight, more preferred 1 to 2.5 percent by weight, of mineralized collagen I are added. The mineralized collagen is preferably produced according to the teachings of U.S. Pat. No. 6,384,196, or U.S. Pat. No. 6,384,197.

After thoroughly mixing the powder with an appropriate amount of an aqueous disodium hydrogen phosphate solution, the product can be further processed as a paste. This cement mixture binds in vivo or in a liquid within four days to a carbonate-containing calcium-deficient hydroxyl apatite (CDHAP).

According to the invention, the admixture of an organic phosphate ester of orthophosphoric acid, for example, phosphoserine (PS) or glycerophosphate (GP) or thiamine pyrophosphate (TP) to the above described basic calcium phosphate bone cement composition leads to an improvement of compressive strength of up to 50 percent and an increase of the specific surface area to a value that is up to 1.5 times that of the bone cement without admixture of an organic phosphate ester.

The organic phosphate ester of the orthophosphoric acid is preferably phosphoserine (orthophospho-l-serine, orthophospho-d-serine, or a mixture of the two stereoisomeres) or glycerophosphate (alpha-glycerophosphate or beta-glycerophosphate) or thiamine pyrophosphate (TP). The organic phosphate ester is added preferably in a quantity of 0.5 to 5 percent by weight, especially preferred more than 1 percent and less than 3 percent by weight.

According to a preferred embodiment of the invention, in place of phosphoserine (PS) or glycerophosphate (GP) or thiamine pyrophosphate (TP), other organic esters of orthophosphoric acid, such as phosphothreonine and phosphotyrosine (preferred are L-phosphothreonine or L-alpha-phosphotyrosine or other steroisomeres), or esters of other polyvalent alcohols are used. According to another embodiment of the invention, as a starting mixture another calcium phosphate bone cement composition is used that contains as a main component (preferably 50 to 65 percent by weight) alpha-tricalcium phosphate and cures to calcium-deficient hydroxyl apatite (CDHAP).

The advantages of the addition of phosphate esters according to the invention are as follows:
a significant increase of the strength, especially compressive strength, of the cured cement;
an increase of the specific surface area;
a finer microstructure;
the initial setting time of the cement paste (determined according to ASTM 266-99) can be varied optimally by means of the ratio of the quantity of the starting mixture relative to the added quantity of liquid;
the cement mixture has no fluctuations of the ion concentration (pH, Ca, P) outside of the physiological range during the binding process;
an improvement of the bone cell activity.

The addition of collagen improves the adhesion of bone cells and increases accordingly the biocompatibility and resorbability, i.e., the conversion of the bone cement to endogenic bone tissue is accelerated. Even though the collagen addition decreases the absolute compressive strength somewhat, it improves the fracture toughness of the material. The collagen addition causes the bone cement according to the invention not to be brittle like a ceramic material but instead to perform like a composite material. The collagen-containing bone cement has, in contrast to pure cement without collagen addition, a certain compressive strength over an extended period of time.

Adding glycerophosphate to the bone cement can also increase the compressive strength. Moreover, glycerophosphate has the advantage that its pharmaceutical harmlessness has already been proven.

The addition of calcium glycerophosphate has an additional advantage in comparison to sodium glycerophosphate in that the calcium concentration available for cells in the vicinity of the bone cement is stabilized; this has a positive effect on the attachment of osteoblast cells. Thiamine pyrophosphate controls also the calcium concentration to a concentration of approximately 2.5 mmol per liter that is optimal for osteoblast cells. The addition of thiamine pyrophosphate leads moreover to an especially fine microstructure of the bone cement.

The following abbreviations are used in the specification:
l/p ratio=ratio of the employed weight of liquid relative to the weight of powder;
D=compressive strength (MPa) after setting for 100 hours in simulated body liquid;
SBF=simulated body fluid;
Asp(d)=specific surface area in m²/g after d days of setting in SBF

### BRIEF DESCRIPTION OF THE DRAWINGS

FIG. 1 shows the measured compressive strength (unit of measure: MPa) for the addition of 34.6 mg sodium glycerophosphate and 69.2 mg sodium glycerophosphate, respectively, in comparison to bone cement without added glycerophosphate.

FIG. 2a shows the measured calcium concentration in mmol per liter of the solution surrounding the bone cement during the setting process as a function of the calcium glycerophosphate contents of the bone cement.

FIG. 2b shows the measured phosphate concentration in mmol per liter of the solution surrounding the bone cement during the setting process as a function of the calcium glycerophosphate contents of the bone cement.

FIG. 3 shows the results of the MTT test (determination of vitality of the cells) as a function of the calcium glycerophosphate contents of the bone cement.

FIG. 4 shows the measured compressive strength (unit of measure: MPa) as a function of the phosphoserine contents of the bone cement.

FIG. 5 shows the measured specific surface area in m²/g as a function of the phosphoserine contents of the bone cement.

FIGS. 6A and 6B show an electron microscope image (magnification: 10,000) of the microstructure of the unmodified bone cement (FIG. 6A) and of the bone cement (FIG. 6B) modified with phosphoserine (25 mg/g) after setting of the bone cement mixtures for four days in simulated body fluid (SBF) (l/p=0.4).

FIG. 7 shows the course of the pH value during the setting process as a function of the phosphoserine contents of the bone cement.

FIG. 8 shows the results of the MTT test (vitality test) as a function of the phosphoserine contents of the bone cement.

FIGS. 9A and 9B show an electron microscope image (magnification: 30,000) of the microstructure of the unmodified bone cement (FIG. 9A) and of the bone cement modified with thiamine pyrophosphate (FIG. 9B) after setting for four days of the cement mixtures in SBF (l/p=0.5).

FIG. 10 shows the calcium concentration in mmol per liter in the solution surrounding the bone cement during the setting process as a function of the thiamine pyrophosphate contents of the bone cement.

### DESCRIPTION OF PREFERRED EMBODIMENTS

Based on the following examples the invention will be explained in more detail.

### EXAMPLE 1

As a base cement for producing a glycerophosphate-containing bone cement, Calcibon®, a calcium phosphate bone cement of the company BIOMET Merck Biomaterials GmbH, Germany, was used. To 1000 g of this base cement, 34.6 mg of β-glycerophosphate (sodium salt; molecular mass 216 g/mol—G1) and 69.2 mg of β-glycerophosphate (sodium salt; molecular mass 216 g/mol—G2) were added, respectively, and thoroughly mixed. As a comparative example, Calcibon® without any additives was prepared (G0).

The mixtures G0, G1, G2 were processed to a paste in accordance with an l/p ratio of 0.28, wherein a 4 percent aqueous disodium hydrogen phosphate solution was used. Subsequently, the pasty cement mixtures were shaped in accordance with their further use.

For determining the compressive strength, cylindrical bodies (diameter 10 mm, height 8 mm) were prepared. They were placed into approximately 5 ml SBF solution and are cured at 37 degrees C. for exactly 100 hours. By means of a materials testing machine—Instron 5566—the critical pressure was determined (advancing speed 8 mm/s) that, relative to the surface of the specimen, provides the compressive strength of the material.

The employed SBF was an aqueous solution of the following salts: 150 mmol/l NaCl; 90 mmol/l NaHCO₃; 1 mmol/l MgSO₄; 1 mmol/l NaH₂PO₄; 5 mmol/l KCl; 1.8 mmol/l CaCl₂; pH=7.4.

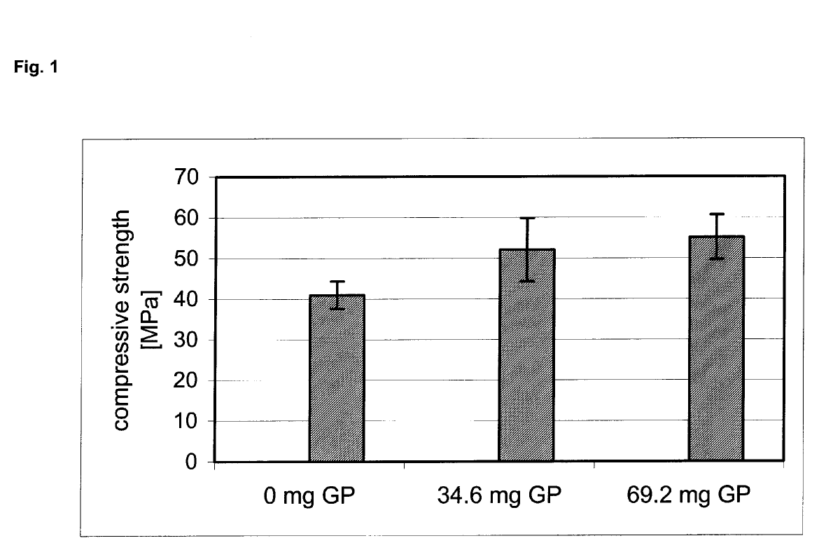

FIG. 1 illustrates the compressive strength for the examples G1 (34.6 mg glycerophosphate) and G2 (69.2 mg glycerophosphate) in comparison to cement without any additives (G0). An increase of the compressive strength with increase of the glycerophosphate proportion is apparent.

For the examples G1 and G2 and the comparative example G0, the measured compressive strength (D) are compiled in the following Table 1.

TABLE 1

| | G0 | G1 | G2 |
|---|---|---|---|
| Na β-glycerophosphate per | 0 mg | 34.6 mg | 69.2 mg |
| g base cement | | | |
| D [MPa] | 41.0 ± 3.4 | 52.1 ± 7.7 | 55.2 ± 5.5 |

The ion concentrations of the solutions surrounding the cement were determined in that during the setting process samples of the solution were taken regularly and analyzed with regard to pH value (glass electrode), calcium contents and phosphate contents (photometric method), in accordance with the procedure described in connection with Example 2. No deviations into cell and tissue damaging pH ranges (pH<7 and pH>8) were measured. The determined calcium and phosphate ion concentrations were also at a level that is well tolerated by cells and tissue.

### EXAMPLE 2

As a base cement, Calcibon®, a calcium phosphate bone cement of the company BIOMET Merck Biomaterials GmbH, Germany, was used. To 1000 mg of this base cement, 16.8 mg calcium glycerophosphate C1 (molecular weight 210 g/mol) was added and thoroughly mixed into the base cement. As a comparative example, Calcibon® without any additives was used (C0).

The substances C0 and C1 were processed to a paste in accordance with an l/p ratio of 0.32, wherein a 4 percent aqueous disodium hydrogen phosphate solution was used. Subsequently, the pasty cement mixtures were shaped in accordance with their further use.

For determining the compressive strength, cylindrical bodies (diameter 10 mm, height 8 mm) were prepared. They were placed into approximately 5 ml SBF solution and cured at 37 degrees C. for exactly 100 hours. By means of a materials testing machine—Instron 5566—the critical pressure was determined (advancing speed 8 mm/s) that, relative to the surface of the specimen, provides the compressive strength of the material.

The employed SBF was an aqueous solution of the following salts: 150 mmol/l NaCl; 4.2 mmol/l NaHCO₃; 1.5 mmol/l MgCl₂; 1 mmol/l K₂HPO₄; 5 mmol/l KCl; 2.4 mmol/l CaCl₂; pH=7.4.

For the examples C0 and C1, the measured compressive strength (D) are compiled in the following Table 2.

TABLE 2

| | C0 | C1 |
|---|---|---|
| Calcium glycerophosphate | 0 mg | 16.8 mg |
| per g base cement | | |
| D [MPa] | 42.3 ± 4.5 | 48.4 ± 4.9 |

The ion concentrations of the solution surrounding the cement were determined in that during the setting process samples of the solution were taken regularly and analyzed with regard to pH value (glass electrode), calcium contents and phosphate contents (photometric method). The photometric method was carried out based on the colored complexes of phosphomolybdate (340 nm, Sigma Diagnostics, method and kit 360-UV) and calcium cresolphthalein (575 nm, Sigma Diagnostics, method and kit 587), respectively.

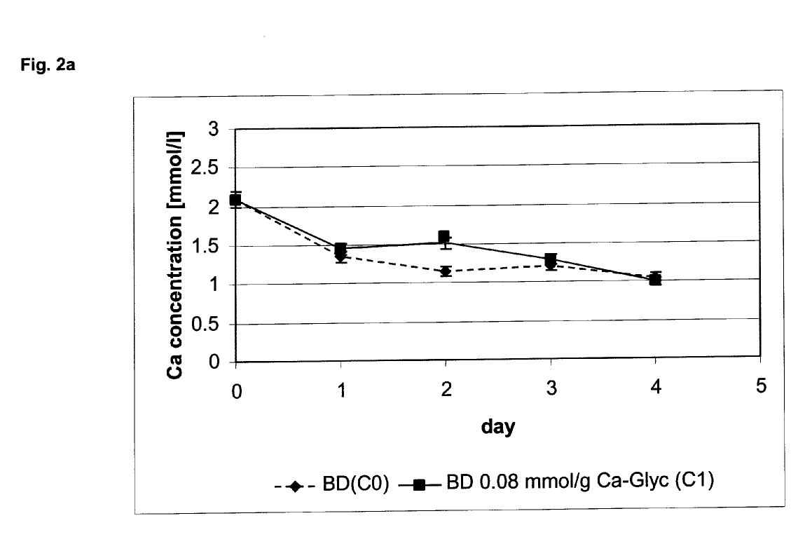

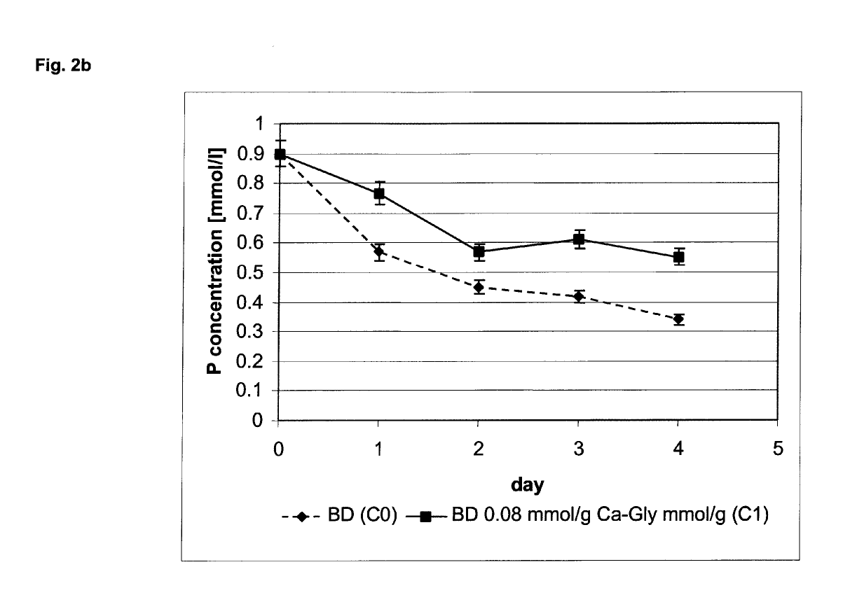

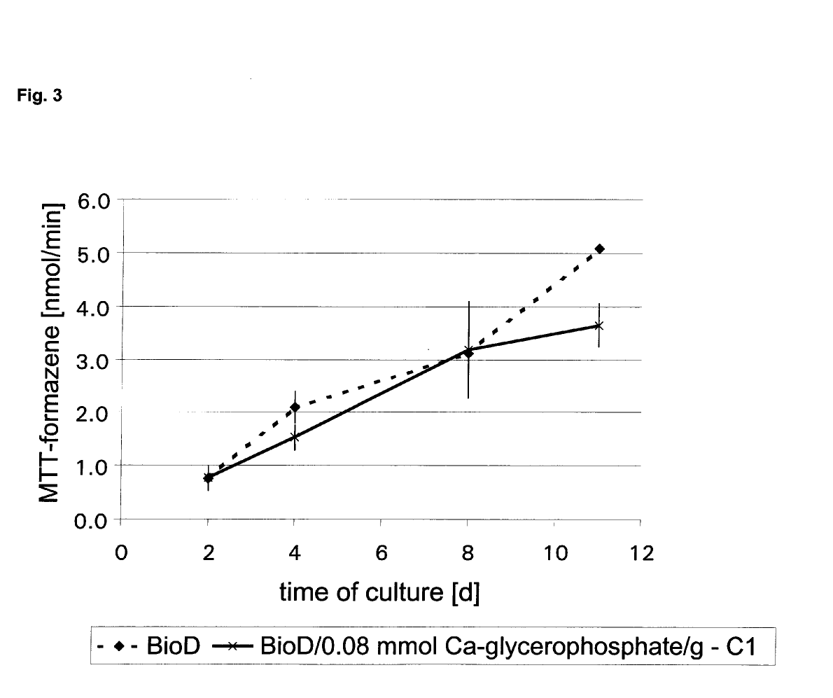

No deviations into cell and tissue damaging pH ranges (pH<7 and pH>8) were measured. The determined calcium and phosphate ion concentrations were also at a level that is well tolerated by cells and tissue. The calcium contents (FIG. 2a) and the phosphate contents (FIG. 2b) of the medium were stabilized by the modified bone cement (C1) at an optimal level for cells. For performing experiments with cells, the bone cement was produced in the form of small platelets (diameter 15 mm, height 1 to 2 mm). These platelets, after setting and drying, were sterilized by using gamma radiation. Before cell culturing, these platelets were pre-incubated in cell culturing medium (DMEM=Dulbecco's Modified Eagle's Medium, containing 10 percent fetal bovine serum) and were then cultured with primary rat calvaria osteoblast cells (12,500 cells per cm²). In order to determine the vitality of the cells as a function of the calcium glycerophosphate of the bone cement, a MTT test (MTT=3-(4,5-dimethylthiazole-2-yl)-2,5-diphenyl tetrazolium bromide) according to the method of T. Mosmann (J. Immunol. Methods, 1983, 65, p 55) was performed. In this connection, the cells were incubated for four hours in MTT. Vital cells formed formazane crystals which were dissolved in a mixture of isopropanol and HCl. The formazane concentration of the solution was determined photometrically at 570 nm. Based on the quantity of formazane generated by the cells, their vitality can be estimated. The result of the MTT test (vitality test) is shown in FIG. 3.

### EXAMPLE 3

As a base cement for producing a phosphoserine-containing bone cement, Calcibon®—a calcium phosphate bone cement of the company BIOMET Merck Biomaterials GmbH, Germany, was used. To 1000 mg of this base cement, 25 mg orthophospho-L-serine (molecular weight 185 g/mol) were added and mixed thoroughly (P1). As a comparative example, Calcibon® without any additives (P0) was used.

The mixtures (P0, P1) were processed to a paste in accordance with an l/p ratio of 0.32, wherein a 4 percent aqueous disodium hydrogen phosphate solution was used. Subsequently, the pasty cement mixtures were shaped depending on their application. The determination of the compressive strength was carried out in accordance with Example 1.

The results of the example P1 and the comparative example P0 for the compressive strength (D) are compiled in Table 3.

TABLE 3

| | P0 | P1 |
|---|---|---|
| phosphoserine per g | — | 25 mg |
| base cement | | |
| D [MPa] | 37.96 ± 4 | 60 ± 7.27 |

A significant increase of the stability after addition of phosphoserine is apparent.

### EXAMPLE 4

As a base cement for producing a phosphoserine-containing and collagen-I-containing bone cement, Calcibon®, a calcium phosphate bone cement of the company BIOMET Merck Biomaterials GmbH, Germany, was used. To an amount of 975 mg of this base cement, 25 mg of mineralized collagen I were added. After thorough homogenization of the base cement and the collagen to a base formulation, 2 mg of orthophospho-L-serine (A1); 10 mg of orthophospho-L-serine (A2); 25 mg of orthophospho-L-serine (A3); and 50 mg of orthophospho-L-serine (A4) were added and thoroughly mixed, respectively. A comparative example (A0) contained no orthophospho-L-serine. The mixtures A0 to A4 were then processed to a paste in accordance with an l/p ratio of 0.42, wherein a 4 percent aqueous disodium hydrogen phosphate solution was used. Subsequently, the pasty cement mixtures were shaped depending on their further use.

For determining the compressive strength cylindrical bodies (diameter 10 mm, height 8 mm) were prepared. They were put into 5 ml SBF solution and cured at 37 degrees C. for exactly 100 hours. By means of a materials testing machine—Instron 5566—the critical pressure was determined (advancing speed 8 mm/s) that, relative to the surface of the specimen, provides the compressive strength of the material.

The employed SBF was an aqueous solution of the following salts: 150 mmol/l NaCl; 90 mmol/l NaHCO₃; 1 mmol/l MgSO₄; 1 mmol/l NaH₂PO₄; 5 mmol/l KCl; 1.8 mmol/l CaCl₂; pH=7.4.

For determining the specific surface areas, the samples, produced according to A1 to A4, were comminuted and the specific surface area of the dry samples was determined by nitrogen adsorption according to the BET method. For this purpose, the surface area analyzer ASAP 2010 was used.

In the following Table 4, the described examples and the determined values for the compressive strength (D) and the specific BET surface area (Asp) are compiled.

TABLE 4

| | A0 | A1 | A2 | A3 | A4 |
|---|---|---|---|---|---|
| phosphoserine per g base | — | 2 mg | 10 mg | 25 mg | 50 mg |
| formulation | | | | | |
| D [MPa] | 28.4 ± 0.3 | 31.9 ± 2.8 | 34.4 ± 3.7 | 41.7 ± 2.9 | 41.7 ± 3.4 |
| Asp (4) [m2/g] | 51.79 ± 0.2 | 68.7 ± 0.3 | 77.9 ± 0.3 | 76.95 ± 0.2 | 37.39 ± 0.2 |
| Asp (30) [m²/g] | 48 | — | — | — | 71.07 ± 0.2 |

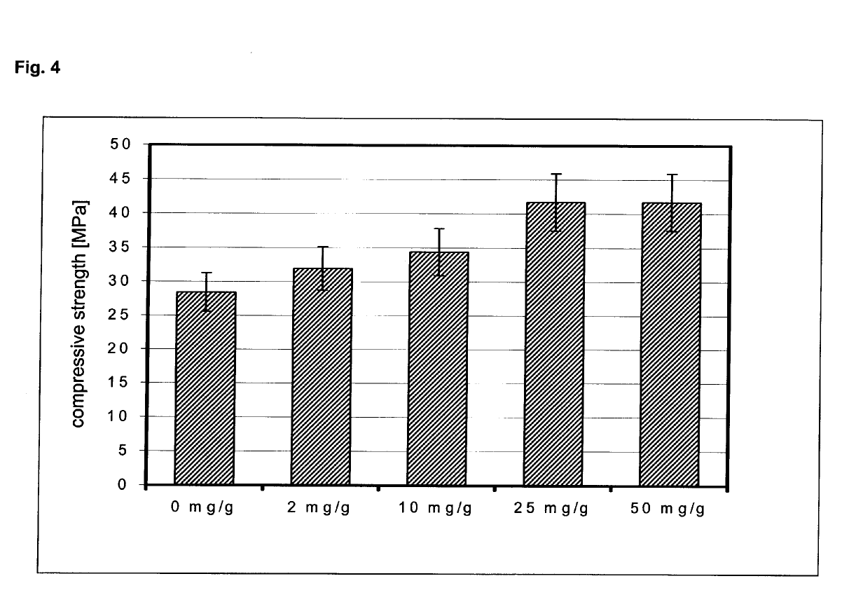

In FIG. 4, the determined values of compressive strength for the samples containing 2 mg, 10 mg, 25 mg, and 50 mg phosphoserine (PS) per g base formulation (examples A1 to A4), respectively, in comparison to the corresponding base formulation A0 (containing collagen I but no phosphoserine) are shown. A significant increase of the stability when increasing the phosphoserine proportion is evident (see also Table 4).

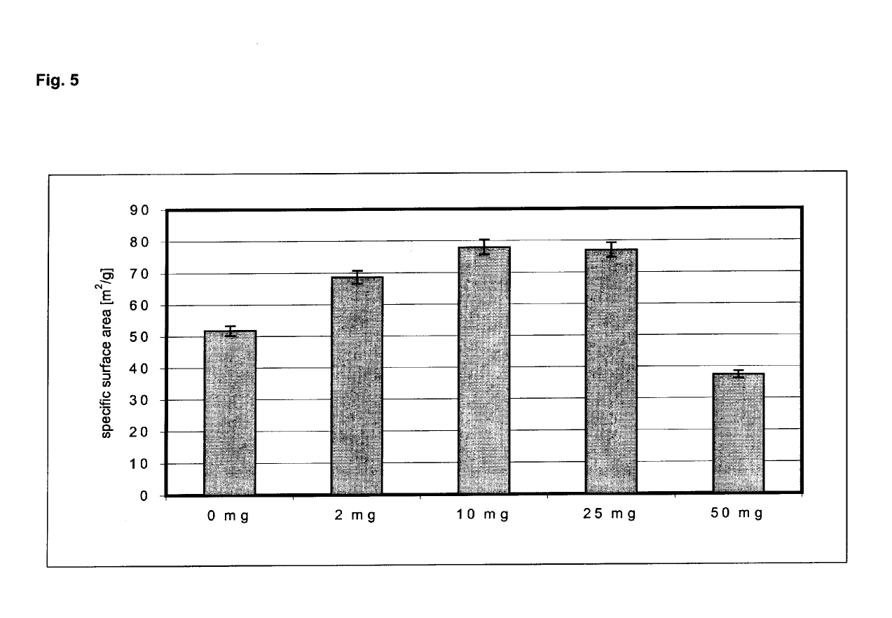

In FIG. 5, the determined BET surface areas as a function of phosphoserine contents of the cement are shown (see also Table 4). A significant increase relative to the comparative base cement A0 (containing collagen I but no phosphoserine) results for the examples A1 to A3 (2 mg to 25 mg phosphoserine per g base formulation). A surface area increase (see also Table 4, last row) for A4 (50 mg phosphoserine per g base formulation) resulted only after approximately 30 days of setting in SBF; this is significantly higher than that for the cement without phosphoserine (48 m²/g).

The employed SBF is an aqueous solution of the following salts: 150 mmol/l NaCl; 90 mmol/l NaHCO₃; 1 mmol/l MgSO₄; 1 mmol/l NaH₂PO₄; 5 mmol/l KCl; 1.8 mmol/l CaCl₂; pH=7.4.

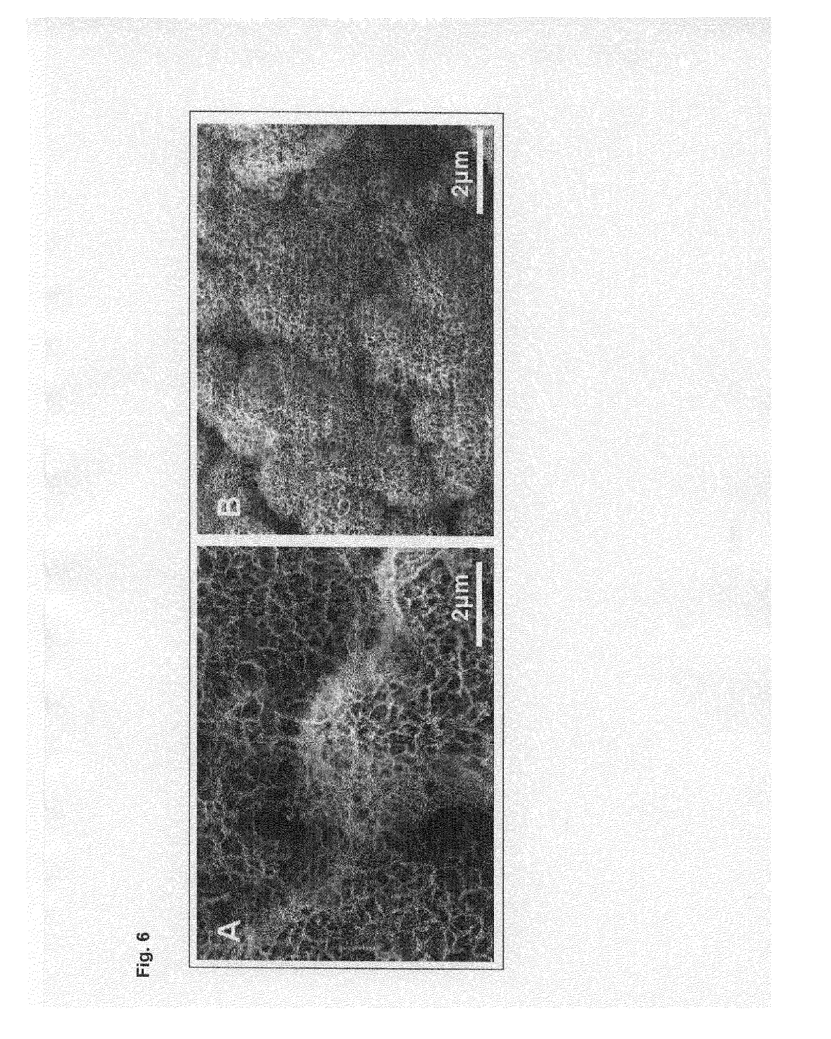

For structural examinations by means of raster electron microscope (REM), specimens of bone cements cured for four days were prepared on aluminum supports and sputtered with carbon (FIGS. 6A, 6B). A comparison of the two images shows that the microstructure of the bone cement A3 with phosphoserine addition (25 mg/g) as shown in FIG. 6B is finer than that of the corresponding bone cement (containing collagen I and not phosphoserine) prepared according to A0 (FIG. 6A).

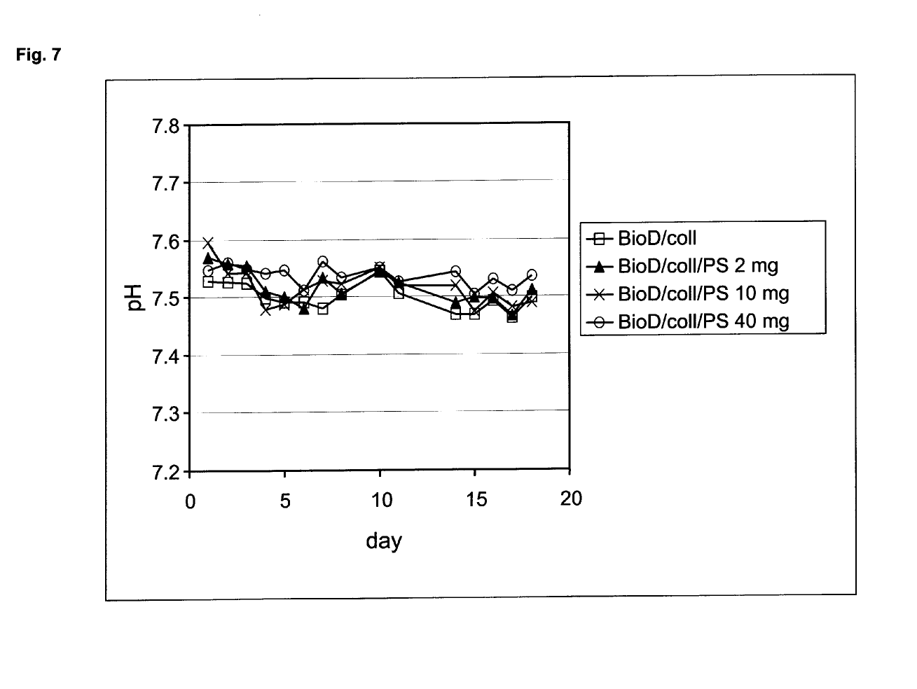

The ion concentration of the solution surrounding the bone cement was determined in that during the setting process regularly samples of the solution were taken and analyzed with regard to pH value (glass electrode), calcium contents and phosphate contents (photometric method). In FIG. 7, as an example the course of the pH value over the course of setting is illustrated. It can be seen that there are no deviations into the cell-damaging and tissue-damaging pH ranges (pH<7 and pH>8). The determined calcium and phosphate ion concentrations were also at a level that is well tolerated by cells and tissue. As a comparison, values for the corresponding bone cement A0 (containing collagen I and no phosphoserine) are shown as BioD/coll.

For carrying out the cell experiments, the bone cement was produced in the shape of small platelets (diameter 50 mm, height 1 to 2 mm). These platelets were sterilized with gamma radiation after setting and drying. Before cell culturing, these platelets were pre-incubated in cell culturing medium (DMEM with 10 percent fetal bovine serum) and then cultured with primary rat calvaria osteoblast cells (12,500 cells per cm²). In order to determine the vitality of the cells as a function of the phosphoserine contents of the bone cement, a MTT test (MTT=3-(4,5-dimethylthiazole-2-yl)-2,5-diphenyltetrazolium bromide) according to the method of T. Mosmann (J. Immunol. Methods, 1983, 65, p 55) was performed. The cells were incubated for four hours in MTT. Vital cells formed formazane crystals which were dissolved in a mixture of isopropanol and HCl. The formazane concentration of the solution was determined photometrically at 570 nm. Based on the quantity of formazane generated by the cells, their vitality can be estimated.

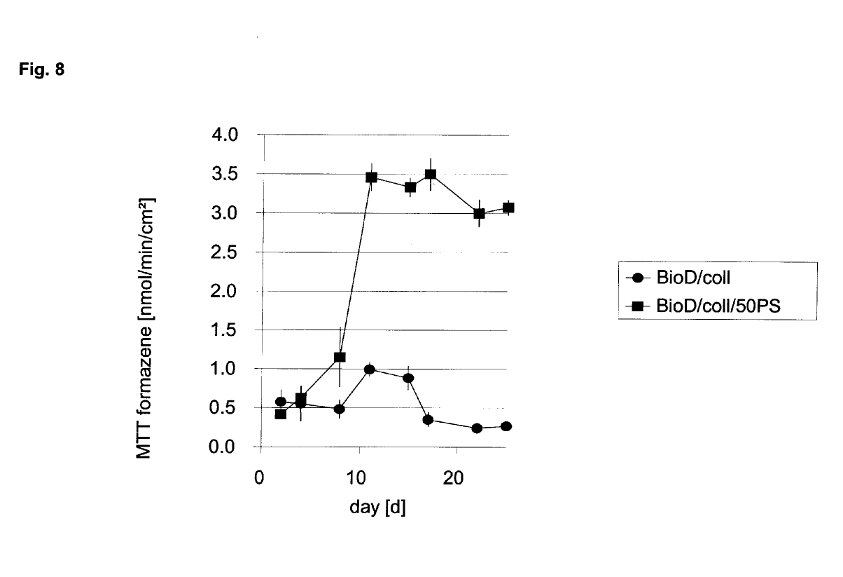

FIG. 8 shows the result of the MTT test (vitality test). It is apparent that the cell vitality of osteoblast cells is improved in the bone cement A4, modified with 50 mg phosphoserine per g base cement, relative to the cell vitality of the osteoblast cells of the cement without phosphoserine. As a comparison, the values for the corresponding bone cement A0 (with collagen I but without phosphoserine) is provided as BioD/coll.

### EXAMPLE 5

As a base cement, Calcibon®, a calcium phosphate bone cement of the company BIOMET Merck Biomaterials GmbH, Germany, was used. To an amount of 1000 mg of this base cement, 36.85 mg of thiamine pyrophosphate (molecular weight 460.8 g/mol) were added (TP1) and thoroughly mixed with the base cement. A comparative sample (TP0) contained no thiamine pyrophosphate. The substances TP0 and TP1 were then processed to a paste in accordance with an l/p ratio of 0.25, wherein a 4 percent aqueous disodium hydrogen phosphate solution was used. Subsequently, the pasty cement mixtures were shaped depending on their further use.

For determining the compressive strength, cylindrical bodies (diameter 10 mm, height 8 mm) were prepared. They were placed into 5 ml SBF solution and cured at 37 degrees C. for exactly 100 hours. The employed SBF was an aqueous solution of the following salts: 150 mmol/l NaCl; 4.2 mmol/l NaHCO₃; 1.5 mmol/l MgCl₂; 1 mmol/l K₂HPO₄; 5 mmol/l KCl; 2.4 mmol/l CaCl₂; pH=7.4.

By means of a materials testing machine—Instron 5566—the critical pressure was determined that, relative to the surface of the specimen, provides the compressive strength of the material. In the following Table 5, the measured values of compressive strength (D) are compiled.

TABLE 5

| | TP0 | TP1 |
|---|---|---|
| thiamine | 0 mg | 36.85 mg |
| pyrophosphate per g | | |
| bone cement | | |
| D [MPa] | 36.45 ± 7 | 55.1 ± 10 |

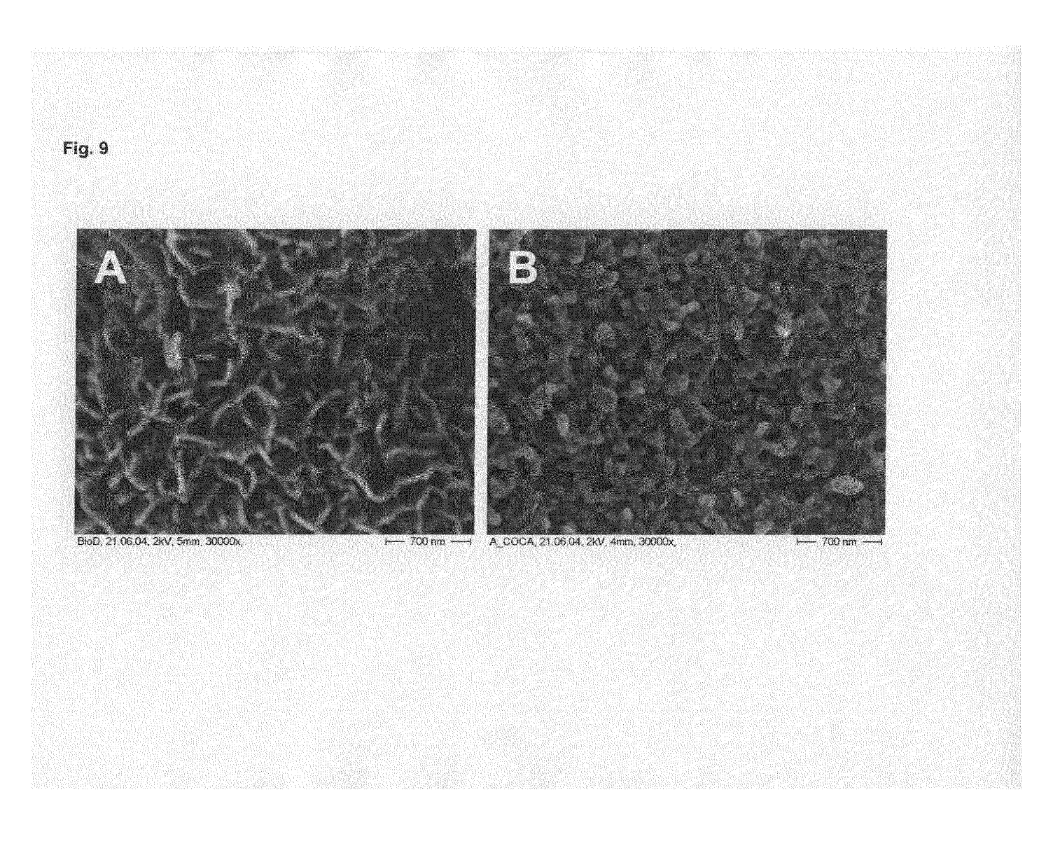

For structural examinations by means of raster electron microscope (REM), specimens of bone cements cured for four days were prepared on aluminum supports and sputtered with carbon (FIGS. 9A, 9B). A comparison of the two images shows that the microstructure of the bone cement with thiamine pyrophosphate addition TP1 (FIG. 9B) is finer than that of the corresponding bone cement (containing no thiamine pyrophosphate) prepared according to TP0 (FIG. 9A).

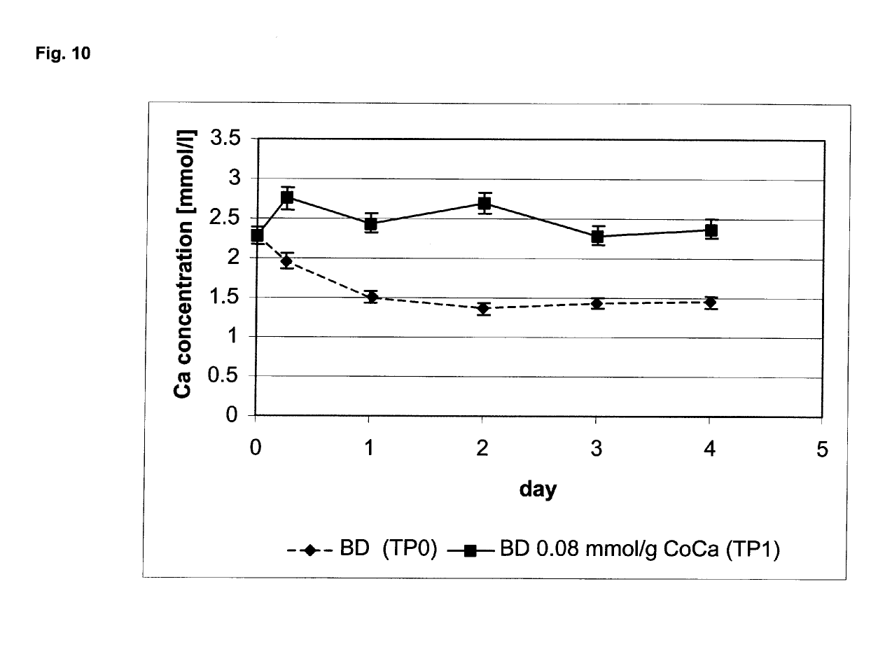

The ion concentration of the solution surrounding the bone cement was determined in that during the setting process regularly samples of the solution were taken and analyzed with regard to pH value (glass electrode), calcium contents and phosphate contents (photometric method) in accordance with Example 2. A deviation into the cell-damaging and tissue-damaging pH ranges (pH<7 and pH>8) was not observed. The determined calcium and phosphate ion concentrations were also at a level that is well tolerated by cells and tissue. The calcium concentration of the surrounding medium was stabilized by the modification of the cement (TP1) to a level of 2-3 mmol/l (see FIG. 10).

1. A calcium phosphate bone cement setting to a calcium-deficient hydroxyl apatite, the calcium phosphate bone cement comprising an organic phosphate ester of orthophosphoric acid or a salt of an organic phosphate ester.

2. A calcium phosphate bone cement according to claim 1, wherein the organic phosphate ester of orthophosphoric acid is a mono phosphate ester.

3. A calcium phosphate bone cement according to claim 1, containing 0.5 to 5 percent by weight of the organic phosphate ester.

4. A calcium phosphate bone cement according to claim 3, containing less than 3 percent by weight of the organic phosphate ester.

5. A calcium phosphate bone cement according to claim 1, wherein the organic phosphate ester is glycerophosphate.

6. A calcium phosphate bone cement according to claim 1, wherein the organic phosphate ester is phosphoserine or thiamine pyrophosphate.

7. A calcium phosphate bone cement according to claim 1, further comprising mineralized collagen I.

8. A calcium phosphate bone cement according to claim 7, containing 0.5 to 5 percent by weight of the mineralized collagen I.

9. A calcium phosphate bone cement according to claim 8, containing 1 to 2.5 percent by weight of the mineralized collagen I.

10. A calcium phosphate bone cement according to claim 1, comprising tricalcium phosphate, dicalcium phosphate (anhydrous), calcium carbonate, and precipitated hydroxyl apatite.
